SEQUENCE LISTING (7 sequences)

SEQ ID NO: 1 - DNA, 29 bp, Artificial Sequence
  Synthetic Polynucleotide
gggaattcgtgctgcaccgccatcaaccg

SEQ ID NO: 2 - DNA, 29 bp, Artificial Sequence
  Synthetic Polynucleotide
ttagagctccagaggcgcagggccaggcg

SEQ ID NO: 3 - DNA, 29 bp, Artificial Sequence
  Synthetic Polynucleotide
acatcaagctttcagaggcgcagggccag

SEQ ID NO: 4 - DNA, 28 bp, Artificial Sequence
  Synethetic Polynucleotide
ggagttccatatgcgccgtacccagcag

SEQ ID NO: 5 - DNA, 28 bp, Artificial Sequence
  Synthetic Polynucleotide
gatagaattcaccggtacggcggcccgc

SEQ ID NO: 6 - DNA, 36 bp, Artificial Sequence
  Synthetic Polynucleotide
cggtcatatgacttcagctgaacctagggcgtatcc

SEQ ID NO: 7 - DNA, 26 bp, Artificial Sequence
  Synthetic Polynucleotide
ggagttccatatgcccatgcttcagg

(19) United States
(12) Patent Application Publication
(10) Pub. No.: US 2009/0081758 A1
(43) Pub. Date: Mar. 26, 2009

(54) Chimeric Cytochrome P450 Proteins and Methods of Use

(75) Inventors: Shengying Li, Ann Arbor, MI (US); David H. Sherman, Ann Arbor, MI (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: THE REGENTS OF THE UNIVERSITY OF MICHIGAN, Ann Arbor, MI (US)

(21) Appl. No.: 12/235,198

(22) Filed: Sep. 22, 2008

Related U.S. Application Data
(60) Provisional application No. 60/974,167, filed on Sep. 21, 2007.

(51) Int. Cl. C12N 9/96

(52) U.S. Cl. 435/188

(57) ABSTRACT

The present invention provides chimeric cytochrome P450 enzymes fused to heterologous reductase domains to generate single-component, self-sufficient, more cost-effective and catalytically more active biosynthetic P450 monooxygenases.

The present application claims benefit of U.S. Provisional Application No. 60/974,167 filed Sep. 21, 2007. The entire text of the aforementioned application is incorporated herein by reference.

This invention was made with U.S. Government support under Research Grant GM-078533 awarded by the National Institutes of Health. The U.S. Government has certain rights in this invention.

### FIELD OF THE INVENTION

The present invention relates to chimeric cytochrome P450 enzymes fused to heterologus reductase domains to generate single-component, self-sufficient, more cost-effective and catalytically more active biosynthetic P450 monooxygenases.

### BACKGROUND OF THE INVENTION

Macrolides are a group of drugs (typically antibiotics) whose bioactivity stems from the presence of a macrolide ring, a large macrocyclic lactone ring, to which one or more deoxy sugars may be attached. The lactone rings are usually 14, 15 or 16-membered rings. Macrolides are a large family of polyketide natural products which include erythromycin, spiramycin, FK506, and avermectin (Katz et al., Polyketide synthesis: Prospects for hybrid antibiotics, Ann. Rev. Microbial. 47: 875-912, 1993; Hopwood, Genetic contributions to understanding polyketide synthases, Chem. Rev. 97: 2465-2497, 1997). Macrolides are classified as polyketides which are secondary metabolites from bacteria, fungi, plants, and animals that are biosynthesized by the polymerization of acetyl and propionyl subunits. Polyketides are the building blocks for a broad range of natural products. A natural product is a chemical compound or substance produced by a living organism found in nature that usually has a pharmacological or biological activity for use in pharmaceutical drug discovery and drug design. Natural products can be considered as such even if it can be prepared by total synthesis. Not all natural products can be fully synthesized and many natural products have very complex structures that are too difficult and expensive to synthesize on an industrial scale.

Polyketides are structurally a very diverse family of natural products with diverse biological activities and pharmacological properties. Polyketide antibiotics, antifungals, cytostatics, anticholesterolemics, antiparasitics, coccidiostatics, animal growth promoters and natural insecticides are in commercial use.

Cytochrome P450 enzymes (P450s) are highly attractive biocatalysts due to their ability to catalyze a variety of regio- and stereo-specific oxidation reactions of complex organic compounds. These reactions occur under mild conditions by taking advantage of the two-electron activated dioxygen that is often challenging in organic synthesis (Ortiz de Montellano, P. R. Cytochrome P450. Structure, Mechanism and Biochemistry, 2nd ed. Ortiz de Montellano P. R., Ed. 1995, New York: Plenum Press. p473; Guengerich, F. P. Chem. Res. Toxicol. 2001, 14, 611). To activate molecular oxygen, redox partners are required to sequentially transfer two reducing equivalents from NAD(P)H to P450 (Hannemann, F. B. A., Ewen K. M., Bernhardt, R. Biochim. Biophys. Acta. 2007. 1770, 330). Classically, there are two major redox partner systems, including an FAD-containing reductase with a small iron-sulfur (Fe₂S₂) redoxin for most bacterial and mitochondrial P450s (Class I), and a single FAD/FMN-containing flavoprotein for eukaryotic microsomal P450s (Class II) (Lewis, D. F. V., Hlavica P. Biochim. Biophys. Acta 2000, 1460, 353; Munro, A. W., Girvan H. M., McLean K. J. Nat. Prod. Rep. 2007, 24, 585). The inherent requirement of cytochrome P450s for separate protein redox partners significantly limits their application in biotechnology.

The discovery of the first self-sufficient P450_{BM3}, which is naturally fused to a eukaryotic-like reductase represents an effective solution to this limitation (Ruettinger, R. T., Fulco, A. J. J. Biol. Chem. 1981, 256, 5728; Otey, C. R., Bandara, G., Lalonde, J., Takahashi, K., Arnold, F. H. Biotechnol. Bioeng. 2005, 93, 494). Self-sufficient cytochrome P450s have an activity that is independent of separate redox partners ferredoxin and ferredoxin reductase. The fusion nature of this enzyme dramatically improves electron transfer efficiency and coupling with the oxidative process, enabling it to be the most efficient P450 enzyme characterized to date (Munro, A. W., Leys D. G., McLean, K. J., Marshall, K. R., Ost, T. W. B., Daff, S., Miles, C. S., Chapman, S. K., Lysek, D. A., Moser, C. C., Page, C. C., Dutton, P. L. Trends Biochem. Sci. 2002, 27, 250). Based upon the self-sufficiency of this naturally fused enzyme, a number of engineered proteins of diverse eukaryotic P450s bearing a reductase domain from P450_{BM3} have been generated with in vitro activities (Fairhead, M., Giannini, S., Gillam, E. M. J.,; Gilardi, G. J. Biol. Inorg. Chem. 2005, 10, 842; Dodhia, V. R., Fantuzzi, A., Gilardi, G. J. Biol. Inorg. Chem. 2006, 11, 903). This work provides ready access to the great catalytic versatility of the membrane-bound eukaryotic P450s. In contrast, the biosynthetic P450s (Class I) lack such a universal reductase that can be used to engineer diverse self-sufficient P450s for either functional identification or potential industrial application.

Recently, a new class of self-sufficient cytochrome P450s exemplified by P450_{RhF} from Rhodococcus sp. NCIMB 9784 was discovered to be naturally fused to a novel FMN/Fe₂S₂ containing reductase partner (De Mot, R., Parret, A. H. A. Trends Microbiol. 2002, 502; Roberts, G. A., Grogan, G., Greter, A., Flitsch, S. L., Turner, N. J. J. Bacteriol. 2002, 184, 3898). Although the physiological function of P450_{RhF} remains unknown, its reductase domain (RhFRED), which is similar to the phthalate family of dioxygenase reductases, is capable of transferring electrons from NADPH to the heme domain of the monooxygenase, supporting 7-ethoxycoumarin dealkylation activity (Roberts, G. A., Celik, A., Hunter, D. J. B., Ost, T. W. B., White, J. H., Chapman, S. K., Turner, N. J., Flitsch, S. L. J. Biol. Chem. 2003, 48914; Hunter, D. J. B., Roberts, G. A., Ost, T. W. B., White, J. H., Muller, S., Turner, N. J., Flitsch, S. L., Chapman, S. K. FEBS Lett. 2005, 579, 2215). Moreover, recent reports from Misawa et al demonstrated that this reductase domain could be used to reconstitute the catalytic activities of various Class I P450s in vivo through expression of corresponding genes fused to RhFRED in Escherichia coli cells (Kubota, M., Nodate, M., Yasumoto-Hirose, M., Uchiyama, T., Kagami, O., Shizuri, Y., Misawa, N. Biosci. Biotechnol. Biochem. 2005, 69, 2421; Nodate, M., Kubota, M., Misawa, N. Appl. Microbiol. Biotechnol. 2006, 71, 455). This observation suggests that RhFRED might be developed into a generally effective redox partner for biosynthetic bacterial P450s. However, the lack of corresponding in vitro data could not unambiguously exclude in trans involvement of additional cellular redox partners.

### SUMMARY OF THE INVENTION

The present invention provides chimeric cytochrome P450 fusion enzymes utilizing a RhFRED fusion strategy to generate single-component, self-sufficient (hence more cost-effective), and catalytically more active biosynthetic P450 monooxygenases compared to native P450 monooxygenases. The invention also provides methods of using the chimeric cytochrome P450 fusion protein to produce natural drug products such as antibiotics.

In certain aspects, the fusion enzyme is a chimeric protein comprising a cytochrome P450 and a heterologous reductase domain, the chimeric protein being self-sufficient and having increased catalytic efficiency compared to native P450 monooxygenases. In various aspects, the chimeric protein catalyzes hydroxylation, epoxidation, or both hydroxylation and epoxidation of a natural product drug substrate.

In one aspect, the substrate is a polyketide. In other aspects, the polyketide is a macrolide. In one embodiment, the cytochrome P450 hydroxylates one or more primary, secondary or tertiary carbon atoms of the substrate. In other embodiments, the cytochrome P450 hydroxylates primary and secondary, secondary and tertiary, or primary and tertiary carbon atoms of the substrate.

In various embodiments, the cytochrome P450 is naturally-occurring and in other embodiments, the cytochrome P450 is non-naturally occurring. In certain aspects, the cytochrome P450 is a bacterial cytochrome P450. a mitochondri alcytochrome P450 or a eukaryotic cytochrome P450. In specific embodiments, the cytochrome P450 is PikC, EryF, MycG or TamI. In one embodiment, the heterologous reductase domain is RhFRED from Rhodococcus sp. NCIMB 9784.

The chimeric protein is further provided in a purified state.

The invention also contemplates a method of producing an antibiotic. The method includes contacting a polyketide natural drug product substrate with the cytochrome P450 chimeric protein under conditions appropriate to produce an antibiotic.

In certain embodiments, the natural drug product substrate is a polyketide. In other embodiments, the polyketide natural drug product substrate is a macrolide. In still other embodiments, the method of producing a natural drug product involves using a chimeric protein that is purified.

### DETAILED DESCRIPTION OF THE INVENTION

Herein is provided the first in vitro characterization of a purified single component bacterial biosynthetic cytochrome P450s fused to a heterologous reductase domain that demonstrates high catalytic efficiency. P450s typically need two redox partners, ferredoxin and ferredoxin reductase, in order to catalyze natural product synthesis reactions through electron transfer. The fusion nature of these chimeric proteins dramatically improves electron transfer efficiency and coupling with the oxidative process, i.e., hydroxylation and/or epoxidation, enabling the chimeric (or fusion) proteins to be more efficient, highly catalytic and more cost-effective P450 enzymes compared to native cytochrome P450s.

Cytochrome P450-mediated electron transport is responsible for oxidative metabolism of both endogenous compounds, which include but are not limited to, fatty acids, steroids, prostaglandins, and exogenous compounds ranging from therapeutic drugs, antibiotics, and environmental toxicants to carcinogens. Vitamins are also potential products envisioned by the invention. For example, both vitamin D₂ and vitamin D₃ are metabolized into prohormones by one or more enzymes located in the liver. The involved enzymes are mitochondrial and microsomal cytochrome P450 (CYP) isoforms, including CYP27A1, CYP2R1, CYP3A4, CYP2J3 and possibly others. These enzymes metabolize vitamin D₂ into two prohormones known as 25-hydroxyvitamin D₂ and 24(S)-hydroxyvitamin D₂, and Vitamin D₃ into a prohormone known as 25-hydroxyvitamin D₃.

The term “cytochrome P450” or simply “P450” is used to encompass any cytochrome P450 enzyme. As such, the P450s encompassed by the present invention include prokayortic and, eukaryotic enzymes. Cytochrome P450 (often abbreviated as CYP, P450, and infrequently CYP450) is a very large and diverse superfamily of hemoproteins which form part of multicomponent electron transfer chains, called P450-containing systems. Known cytochrome P450s amenable to the invention include the CYP1 family ( CYP1A1; CYP1A2; CYP1B1), the CYP2 family (CYP2A6; CYP2A7, CYP2B6, CYP2A13; CYP2B6; CYP2C8; CYP2C9; CYP2C18, CYP2C19; CYP2D6; CYP2E1; CYP2F1; CYP2J2; CYP2R1; CYP2S1; CYP2U1, CYP2W1), the CYP3 family ( CYP3A4; CYP3A5; CYP3A7; CYP3A43), the CYP4 family (CYP4A11; CYP4A22; CYP4B1; CYP4F2, CYP4F3, CYP4F8, CYP4F11, CYP4F12, CYP4V2, CYP4Z1), CYP5A1, CYP7A1, CYP7B1, CYP8A1, CYPS8B1, CYP11A1, CYP11B1, CYP11B2, CYP17A1, CYP19A1, CYP20A1, CYP21A2, CYP24A1, CYP26A1, CYP26B1, CYP26C1, CYP27A1, CYP27B1, CYP27C1, CYP39A1, CYP46A1, and CYP51A1.

In its simplest form, the P450 monooxygenase system consists of NAD(P)H cytochrome P450 reductase (CPR; NAD(P)H-ferrihemoprotein reductase) and one of many cytochrome P450 enzymes listed above. Both CPR and cytochrome P450 are integral membrane proteins, and CPR is one of only two known mammalian enzymes containing both FMN (flavin mononucleotide or riboflavin-5′-phosphate) and FAD (flavin adenine dinucleotide) as prosthetic groups, the other being various isoforms of nitric-oxide synthase (NOS). Other physiological electron acceptors of CPR include microsomal heme oxygenase (Schacter, B. A., Nelson, E. B., Marver, H. S. & Masters, B. S. S. 1972, J. Biol. Chem. 247, 3601-3607.) and cytochrome b5 (Enoch, H. G. & Strittmatter, P. 1979, J. Biol. Chem. 254, 8976-8981.) and, although nonphysiological, CPR is capable of transferring reducing equivalents to cytochrome c (Horecker, B. L. 1950, J. Biol. Chem. 183, 593-605).

Consequently, the term “reductase domain” encompasses any reductase function (i.e., electron donation). Reductase domains include bacterial, microsomal, mitochondrial, fungal, eukaryotic, pant and animal reductase domains. In certain embodiments, reductase domains contemplated by the invention include, but are not limited to, NAD(P)H-ferrihemoprotein reductase, ferredoxin, ferredoxin reductase, flavodoxin, flavodoxin reductase, putidaredoxin, and putidaredoxin reductase.

The term “natural product” refers to is a chemical compound or substance produced by a living organism found in nature that usually has a pharmacological or biological activity.

The term “polyketide” encompasses a large class of diverse compounds that are characterized by more than two carbonyl groups connected by single intervening carbon atoms that are produced by bacteria, fungi, plants and animals. Polyketides include various substances having antibiotic, anticancer, cholesterol-lowering, or immunosuppressive effects. Polyketides include antibiotics such as tetracyclines, macrolides (such as erythromycin or pikromycin) and non-macrolides (such as tirandamycin or curacin), anticancer agents such as daunomycin, immunosuppressants such as FK506, tacrolimus, ascomycin, and rapamycin, and antifungals and antibacterials such as linearmycin A and lienomycin. Polyketides also include, but are not limited to, avermectin, nemadectin, candicidin, niddamycin, oleandomycin, narbomycin, rifamycin, and spiramycin. As will be appreciated by those skilled in the art, a wide variety of domains, modules, protein subunits as well as whole proteins are available from known polyketide synthase biosynthetic clusters that can be used to make alterations in the biosynthesis of a polyketide and/or a macrolide.

The term “macrolide” is a compound with a structure that contains a macrocyclic lactone ring to which one or more deoxy sugars may be attached. These compounds encompass any macrolide antibiotic, and include, for example and without limitation, a group of antibiotics produced by various strains of Streptomyces that have a complex macrocyclic structure. The lactone rings are usually 14, 15 or 16-membered rings. Macrolide antibiotics include, but are not limited to, erythromycin A, B and C, azithromycin, griesomycin, methymycin, narbomycin, neomethymycin, oleandomycin, pikromycin, plicacetin, carbomycin A and B, spriamycin I, II and III, leucomycin, mycinamicin, rapamycin, clarithromycin, FK506 (tacrolimus), FK520 (ascomycin or immunomycin), antascomicin, meridamycin, FK520, hyg, FK523, meridamycin, antascomicin, FK525, tsukubamycin, ivermectin, milbemycin D, sorapheni A, vancomycin, teicoplanin, roxithromycin, josamycin, ristocetin, actinoidin, avoparcin, actaplanin, teichomycin and telithromycin. Synthetic macrolide genes may comprise one or more of angMIII, angMI, angB, angAI, angAII, angorfl4, angorf4, tylMIII, tylMI, tylB, tylAI, tylAII, eryCVI, spnO, eryBVI, eryK, tyl Ia and cry G, eryCIII, tylMII, angMII, desVII, eryBV, spnP and midI.

In addition to macrolide and non-macrolide polyketides, the invention contemplates production of all antibiotics whose biosyntheses involve cytochrome P450 catalyzed step(s). These antibiotics include, but are not limited to, tylosin, cryptophycin, rebeccamycin, oleandomycin, epothilone, rhizoxin, erythromycin, mycinamicin, and tirandamycin.

The term “self-sufficiency” means the activity of cytochrome P450 is independent of separate redox partners, such as and without limitation, ferredoxin and ferredoxin reductase.

The term “purified” as used herein refers to a protein composition that has been isolated under conditions that reduce or eliminate the presence of unrelated materials, i.e., contaminants, including native materials from which the material is obtained. For example, a purified enzyme is preferably substantially free of host cell or culture components, including tissue culture or egg proteins, non-specific pathogens, and the like. In one embodiment, purified material substantially free of contaminants is at least 50% pure; at least 60%, at least 70%, at least 80%, at least 90%, at least 95% or even at least 99% pure. Purity can be evaluated by chromatography, gel electrophoresis, immunoassay, composition analysis, biological assay, and other methods known in the art.

In one embodiment of the invention, the purification of the P450 chimeric fusion enzyme is accomplished using previously developed procedures with minor modifications (Xue, Y., Wilson, D., Zhao, L., Liu, H.-w., Sherman, D. H. Chem. Biol. 1998, 5, 661-667; Li, S., Podust, L. M., and Sherman, D. H. J. Am. Chem. Soc. 2007, 129, 12940-12941; Anzai, Y., Li, S., Chaulagain, M. R., Kinoshita, K., Kato, F., Montgomery, J., and Sherman D. H. Chem. Biol. In Press). Cells transfected with desired plasmids are grown and harvested by centrifugation. The cell pellet is resuspended and lysis is accomplished using a sonic dismembrator. The insoluble material is then separated by centrifugation and the soluble fraction is collected and loaded onto a Ni-NTA column, whereby the contaminants are removed by wash buffer. The elution buffer is added onto the column, and eluted protein fraction is concentrated and desalted by buffer exchange with a PD-10 column.

In another aspect, purification of the P450 chimeric fusion enzyme is accomplished using various purification methods known in the art including, but not limited to, affinity chromatography, (Yasukochi Y and Masters B S S 1976, J. Biol. Chem. 251: 5337-5344), nickel-nitrotriacetic acid column chromatography (Helvig, C. Koener, J. F., Unnithan, G. C. and Feyereisen, R. 2004, PNAS 101:4024-4029), aminooctyl Sepharose 4B column chromatography (Matsunaga et al., Drug Metab Dispos. 1998, (10):1045-7), phenyl-Sepharose hydrophobic interaction column (Funhoff, E. G. et al. J Bacteriol. 2006 188(14): 5220-5227), size exclusion high performance liquid chromatography (Nakhgevany, R. et al. Biochem. Biophys. Res. Comm. 1996, (222) 406-409) cation exchange chromatography, anion exchange chromatography (Yasukochi, Y., Okita, R. T. & Masters, B. S. S. 1980, Arch. Biochem. Biophys. 202, 491-498), hydroxylapatite chromatography and DEAE-Sepharose CL-6B column chromatography (Sasaki, M. et al., Applied and Environmental Microbiology 2005, (71) 8024-8030). Also contemplated by the invention are purification procedures such as differential centrifugation, solubilization of CHAPS, protein precipitation by PEG precipitation and DE-32 column chromatography (Cai-Hong Yu and Xi-Wu Gao Insect Science 2005, (12) 313-317).

Methods of producing an antibiotic using a cytochrome P450 chimeric fusion protein are contemplated by the invention. In one embodiment, the method involves contacting a macrolide or polyketide natural product substrate with a P450 chimeric fusion protein under conditions that permit the fusion protein to catalyze a reaction that produces an antibiotic. The chimeric P450 fusion protein is either purified or non-purified. By way of example, antibiotic production includes, but is not limited to, pikromycin: A purified PikC-RhFRED cytochrome P450 fusion protein of the invention is used to cataylze the hydroxylation of narbomycin to produce the antibiotic pikromycin. Confirmation of transformation can then be achieved by HPLC trace, mass spectrometry, UV spectrum analysis, or other methods known in the art.

### PikC

The PikC cytochrome P450 in this study is involved in the pikromycin biosynthetic pathway of Streptomyces venezuelae (Xue, Y., Zhao, L., Liu, H.-w., Sherman, D. H. Proc. Natl. Acad. Sci. U.S.A. 1998, 95, 12111). PikC catalyzes the final hydroxylation step toward both the 12-membered ring macrolactone YC-17 and the 14-membered ring macrolactone narbomycin to produce methymycin/neomethymycin and pikromycin as major products (Xue, Y., Wilson, D., Zhao, L., Liu, H.-w., Sherman, D. H. Chem. Biol. 1998, 5, 661; Lee, S. K., Park, J. W., Kim, J. W., Jung, W. S., Park, S. R., Choi, C. Y., Kim, E. S., Ahn, J. S., Sherman, D. H., Yoon, Y. J. J. Nat. Prod. 2006, 69, 847).

Recently, the structural basis was elucidated for the remarkable substrate flexibility by analyzing ligand-free and substrate-bound structures of PikC (Sherman, D. H., Li, S., Yermalitskaya, L. V., Kim, Y., Smith, J. A., Waterman, M. R., Podust, L. M. J. Biol. Chem. 2006, 281, 26289). However, since the native redox partner of PikC remains unknown, its in vitro activity has depended on expensive spinach ferredoxin reductase (Fdr) and ferredoxin (Fdx), as are many other biosynthetic P450s (Lambalot, R. H., Cane, D. E. Biochemistry 1995, 34, 1858; Andersen, J. F., Hutchinson, R. C. J. Bacteriol 1992, 174, 725; Ogura, H., Nishida, C. R., Hoch, U. R., Perera, R., Dawson, J. H., Ortiz de Montellano, P. R. Biochemistry 2004, 43, 14712). To investigate an alternative electron transfer pathway mimicking the fusion organization in P450RhF, the PikC gene was linked to the RhFRED gene.

### EryF

Cytochrome P450 EryF, isolated from the actinomycete bacterium Saccharopolyspora erythraea, is responsible for the biosynthesis of the antibiotic erythromycin by C6-hydroxylation of the macrolide 6-deoxyerythronolide B (DEB). Following hydroxylation, the 6-DEB macrolide ring is further elaborated to erythromycin A (Lambalot, R. H., Cane, D. E. Biochemistry 1995, 34, 1858; Andersen J. F., Tatsuta K., Gunji H., Ishiyama T., Hutchinson C. R. Biochemistry 1993, 32, 1905-1913; Andersen, J. F., Hutchinson, R. C. J. Bacteriol. 1992, 174, 725; Ogura, H., Nishida, C. R., Hoch, U. R., Perera, R., Dawson, J. H., Ortiz de Montellano, P. R. Biochemistry 2004, 43, 14712). When RhFRED was fused to another prototype biosynthetic P450 EryF, a more active self-sufficient biocatalyst was obtained once again.

### MycG and TamI

MycG, one P450 monooxygenase from the mycinamicin biosynthetic pathway is able to either hydroxylate or epoxidize mycinamicin IV (M-IV) leading to mycinamicin V (M-V) or mycinamicin I (M-I), respectively. M-V can be further epoxidized by MycG resulting in final product mycinamicin II (M-II) (Anzai, Y., Li, S. et al. Chem. Biol. In Press). TamI was recently characterized as another versatile P450 enzyme, which is capable of catalyzing multiple hydroxylation and epoxidation reactions, thus leading to remarkable post-PKS-NRPS structural diversification in tirandamycin biosynthesis (Carlson C. J., Li, S. et al. unpublished).

The invention will be more fully understood by reference to the following examples which detail exemplary embodiments of the invention. They should not, however, be construed as limiting the scope of the invention. All citations throughout the disclosure are hereby expressly incorporated by reference.

### EXAMPLES

### Example 1

### Molecular Cloning

The hybrid PikC-RhFRED gene was cloned into pET28b(+), and overexpressed in E. coli BL21 (DE3) to generate N-terminal His₆-tagged PikC-RhFRED. After Ni-NTA chromatography, the purified red-colored recombinant P450 displayed (upon reduction) the signature peak at 450 nm in the CO-difference spectrum. Interestingly, gel filtration chromatography indicated that PikC-RhFRED predominantly dimerizes in storage buffer solution containing 0.2 mM dithioerythritol (DTE). In contrast, wild type (wt) PikC was shown to be monomeric under the same conditions. It was thus unclear whether the inter-monomer electron transfer could occur in the dimeric PikC-RhFRED as in P450_{BM3} (Neeli, R., Girvan, H. M., Lawrence, A., Warren, M. J., Leys, D., Scrutton, N. S., Munro, A. W. FEBS Lett. 2005, 579, 5582).

The gene encoding the reductase domain of P450_{RhF} (RhFRED) and the linker sequence was amplified by PCR under standard conditions using forward primer: 5′-GGGAATTCGTGCTGCACCGCCATCAACCG-3′ (the italic bases represent EcoRI restriction site), reverse primer 1: 5′-TTAGAGCTCCAGAGGCGCAGGGCC AGGCG-3′ (the SacI cutting site is underlined) for amplifying RhFRED gene without the stop codon, and reverse primer 2: 5′-ACATCAAGCTTTCAGAGGCGC AGGGCCAG-3′ (the HindIII cutting site is underlined) for cloning RhFRED gene retaining the stop codon. The cDNA without and with the stop codon were digested by NdeI/SacI and NdeI/HindIII restriction enzyme pairs, respectively, and then ligated into the NdeI/SacI digested pET21b(+) (Novagen) and NdeI/HindIII digested pET28b(+) (Novagen) correspondingly, generating the recombinant plasmid pET21b(+)-RhFRED and pET28b(+)-RhFRED. On the other hand, using previously prepared plasmid pET28a-pikC (Xue, Y.; Wilson, D.; Zhao, L.; Liu, H.-w.; Sherman, D. H. Chem. Biol. 1998, 5, 661) as template, the pikC gene with stop codon removed was amplified by PCR under standard conditions using a pair of primers as follow: forward, 5′-GGAGTTCCATATGCGCCGTACCCAGCAG-3′, reverse: 5′-GATAGAATTCACCGGTAC GGCGGCCCGC-3′ (The italic and underlined bases represent the introduced NdeI and EcoRI restriction sites for following cloning manipulation). The NdeI/EcoRI double digested pikC gene was then ligated into the previously NdeI/EcoRI-digested pET21b(+)-RhFRED and pET28b(+)-RhFRED to generate vectors pET21b(+)-pikC-RhFRED and pET28b(+)-pikC-RhFRED for overexpressing C-His₆-tagged and N-His₆-tagged fusion protein PikC-RhFRED.

Using cosmid pMRO1 (for mycG) and plasmid pSJ2-tamI (for tamI) as templates, the mycG and tamI genes with stop codon removed were amplified by PCR under standard conditions using primers as follow: forward, 5′-CGGTCATATGACT TCAGCTGAACCTA GGGCGTATCC-3′ for mycG, 5′ -GGAGTTCCATTTGCCCA TGCTTCAGG-3′ for tamI (the italic letters represent the introduced NdeI site); reverse, 5′-GCGCGAATTCCCACACGA CCGGCAGCTCGAGT-3′ for mycG, 5′-ACATCGAATTCTGGGCGGTTCAGCCG-3′ for tamI (the underlined bases represent the introduced EcoRI cutting site). The gel purified cDNAs were rescued by double digestion of NdeI and EcoRI. Then, the fragments containing mycG and tamI genes were ligated into the NdeI/EcoRI-digested pET28b-pikC-R FRED as previously reported (J. Am. Chem. Soc. 2007, 129, 12940-12941) to generate recombinant plasmids pET28b-mycG-RhFRED and pET28b-tamI-RhFRED, respectively. The accuracy of recombinant gene was confirmed by DNA sequencing.

The preceding experimental procedures including protein overexpression and purification, CO-bound reduced difference assay, P450 in vitro assay, HPLC and LC-MS analysis follow previous reports (J. Am. Chem. Soc. 2007, 129, 12940-12941; Anzai, Y., Li, S. et al. Chem. Biol. In Press; Carlson C. J., Li, S. et al unpublished), correspondingly.

### Example 2

### Overexpression and Purification of Fusion Proteins

Overexpression and purification of diverse P450-RhFRED followed previously developed procedures with minor modifications (Xue, Y., Wilson, D., Zhao, L., Liu, H.-w., Sherman, D. H. Chem. Biol. 1998, 5, 661-667; Li, S., Podust, L. M., and Sherman, D. H. J. Am. Chem. Soc. 2007, 129, 12940-12941; Anzai, Y., Li, S., Chaulagain, M. R., Kinoshita, K., Kato, F., Montgomery, J., and Sherman D. H. Chem. Biol. In Press). The E. coli BL21 (DE3) transformants carrying certain plasmids were grown at 37° C. in 1 liter of LB broth containing thiamine (1 mM), 5% glycerol, 50 μg/ml of kanamycin, and a rare salt solution (6750 μg/l FeCl₃, 500 μg/l ZnCl₂, CoCl₂, Na₂MoO₄, 250 μg/l CaCl₂, 465 μg/l CuSO₄, and 125 μg/l H₃BO₃) until OD₆₀₀ reached 0.6-0.8. Then isopropyl β-_{D}-thiogalactoside (IPTG, 0.1 mM) and δ-aminolevulinic acid (1 mM) were added, and the cells were cultured at 18° C. overnight. After harvesting cells by centrifugation, 40 ml of lysis buffer (50 mM NaH₂PO₄, pH8.0, 300 mM NaCl, 10% glycerol, 10 mM imidazole) was used to resuspend the cell pellet. Lysis was accomplished on a Model 500 Sonic Dismembrator (ThermoFisher Scientific). The insoluble material was separated by centrifugation (35,000×g, 30 min at 4° C.). The soluble fraction was collected and incubated for 1 h at 4° C. after addition of 1 ml Ni-NTA resin. The slurry was loaded onto an empty column, and the column was then washed with 40 to 80 ml of wash buffer (50 mM NaH₂PO₄, pH8.0, 300 mM NaCl, 10% glycerol, 20˜30 mM imidazole). The elution buffer (50 mM NaH₂PO₄, pH8.0, 300 mM NaCl, 10% glycerol, 250 mM imidazole) was added onto the column, and eluted protein fraction was concentrated with Amicon Ultra 4, Ultracel—50K (Millipore). Subsequent desalting was attained by buffer exchange into desalting buffer (50 mM NaH₂PO₄, pH7.3, 1 mM EDTA, 0.2 mM dithioerythritol, 10% glycerol) with a PD-10 column (GE Healthcare).

SDS-PAGE analysis of functional N-terminal His₆-tagged PikC-RhFRED revealed a molecular mass of 83 kDa. Using gel filtration analysis, the deduced molecular mass of wt-PikC and PikC-RhFRED was approximately 55 kDa and 150 kDa, respectively, suggesting the monomeric (wt) and dimeric (fusion) form in 50 mM sodium phosphate pH 7.3 buffer containing 300 mM NaCl, 10% glycerol.

UV-visible absorption spectra for purified N-terminal His₆-tagged PikC-RhFRED was also performed. This assay was employed to also determine the concentration of functional P450 enzyme using the extinction coefficient of 91,000 M⁻¹·cm⁻¹ (Omura, T., Sato, R. J. Biol. Chem. 1964, 239, 2379).

### Example 3

### Spectral Substrate Binding Assays

Spectral substrate binding assays were performed using a UV-visible spectrophotometer 300 Bio (Cary) at room temperature by titrating 30 mM substrate DMSO solution (blank DMSO for reference group) into 1 ml of 1 μM P450 sample in 1 μl aliquots, leading to substrate concentration ranging from 30 to 360 μM. The series of Type I difference spectra were used to deduce ΔA (Aₚₑₐₖ₍₃₈₉₎−A_{trough(424)}). Then, the data were fit to Michaelis-Menten equation to obtain the dissociation constant K_{d}. As mentioned above, one benefit of the fusion protein arrangement is the facility of the covalent linkage to stabilize productive interactions between the P450 and redox partner, thus enhancing electron transfer efficiency. As such, this fusion may improve the catalytic activity in terms of k_{cat}, whereas the substrate specificity would not be changed significantly (Munro, A. W., Girvan, H. M., McLean, K. J. Biochim. Biophys. Acta. 2007, 1770, 345; Yabusaki, Y. Biochimie 1995, 77, 594). To test whether this also applies to PikC-RhFRED, we first determined the substrate binding affinity of YC-17 and narbomycin toward both PikC and PikC-RhFRED. As expected, YC-17 and narbomycin bind to PikC-RhFRED with K_{d} values of 92.6±0.5 μM and 215.0±4.2 μM, respectively, which are similar to 112.9±1.9 μM (YC-17) and 288.3±7.1 μM (narbomycin) toward wt-PikC. This indicates that attachment of the heterologous reductase domain has no significant impact on substrate binding to PikC.

### Example 4

### HPLC Analysis of Reactions Catalyzed by PikC, MycG and TamI Enzymes

The ability of PikC-RhFRED to hydroxylate YC-17 and narbomycin in vitro was tested when electron donor NADPH was provided. This chimeric protein showed significantly improved catalytic activity compared to wt-PikC in the presence of exogenous redox partners (spinach Fdr and Fdx), producing higher yields of methymycin/neomethymycin and pikromycin under identical reaction conditions.

After developing an RhFRED fusion strategy to generate single-component, self-sufficient (hence more cost-effective), and catalytically more active biosynthetic P450 monooxygenases including PikC-RhFRED and EryF-RhFRED (Li, S., Podust, L. M., and Sherman, D. H., J. Am. Chem. Soc. 2007, 129, 12940-12941), whether this strategy also applied to other more biosynthetic cytochrome P450 enzymes was determined. The RHFRED reductase domain was fused to two more biosynthetic P450 enzymes including MycG and TamI involved in mycinamicin and tirandamycin biosynthetic pathway, respectively.

The reactions for MycG and TamI enzymes were also carried out using previously developed assays with minor modifications (Xue, Y.; Wilson, D.; Zhao, L.; Liu, H.-w.; Sherman, D. H. Chem. Biol. 1998, 5, 661). The PikC assay contained 1 μM PikC-RhFRED (or 1 μM PikC with 3.5 μM spinach ferredoxin and 0.1 U/ml spinach ferredoxin-NADP⁺ reductase), 200 μM YC-17 or narbomycin, and 0.5 mM NADPH in 100 μl of desalting buffer (50 mM NaH₂PO₄, pH 7.3, 1 mM EDTA, 0.2 mM dithioerythritol, 10% glycerol). The reaction was stopped and extracted after 1 h of incubation at 30° C. by addition of 2×200 μl of chloroform. The extracts were dried, dissolved in 120 μl of methanol and analyzed by Xbridge™ C18 5 μtm 250 mm reverse-phase column at 230 nm, using 10-70% solvent B (A: deionized water +10 mM ammonium acetate, B: acetonitrile) at 1 ml/min over 30 min. The peak identity in each HPLC trace was determined by mass spectrometry and comparison with authentic compounds regarding HPLC retention time and U spectrum.

Upon fusion to RhFRED, both MycG and TamI fusion P450 enzymes showed unambiguous self-sufficiency and slightly increased activity compared to the wild type enzymes when partnered by spinach ferredoxin (fdx) and spinach ferredoxin reductase (fdr).

These results confirm that PikC-RhFRED, MycG-RhFRED and TamI-RhFRED are self-sufficient P450 enzymes. The pET21b(+)-pikC-RhFRED was prepared and the purified C-terminal His₆-tagged PikC-RhFRED was isolated. This protein showed a similar CO-difference spectrum as its N-terminal His₆-tagged counterpart (Data not shown). However, it lacked catalytic activity, which is consistent with a similar C-terminal His₆-tagged form of original P450_{RhF} (De Mot, R., Parret, A. H. A. Trends Microbiol. 2002, 502; Roberts, G. A., Grogan, G., Greter, A., Flitsch, S. L., Turner, N. J. J. Bacteriol. 2002, 184, 3898). This result provides additional evidence for the importance of the C terminus of RhFRED for electron transfer.

### Example 5

### Steady-State Kinetics

Subsequently, the kinetic parameters of PikC-RhFRED were compared with those of the PikC-Fdr-Fdx three component system. As previously reported, (Xue, Y., Wilson, D., Zhao, L., Liu, H.-w., Sherman, D. H. Chem. Biol. 1998, 5, 661; Lee, S. K., Park, J. W., Kim, J. W., Jung, W. S., Park, S. R., Choi, C. Y., Kim, E. S., Ahn, J. S., Sherman, D. H., Yoon, Y. J. J. Nat. Prod. 2006, 69, 847; Graziani, E. I., Cane, D. E., Betlach, M. C., Kealey, J. T., McDaniel, R. Bioorg. Med. Chem. Lett. 1998, 3117) substrate inhibition was observed in all cases when its concentration was greater than 250 μM. Moreover, the solubility limitation (less than 500 μM) of macrolides in aqueous solution prevented us from deducing the K, value. Therefore, the apparent specificity constants (k_{cat}/Kₘ) were determined by fitting the low-concentration data to the linear region of a Michaelis-Menten curve. By directly monitoring the substrate consumption by HPLC, the k_{cat}/Kₘ values of PikC-RhFRED were determined to be 0.96 and 1.20 μM⁻¹·min⁻¹ for YC-17 and narbomycin, respectively. In contrast, the specificity constants of wt-PikC partnered by Fdr and Fdx were 0.24 μM⁻¹·min⁻¹ for YC-17 and 0.31 μM⁻¹·min⁻¹ for narbomycin. It is evident that the fusion enhanced the catalytic activity approximately 4 fold for both YC-17 and narbomycin. Notably, the kinetic parameters of wt PikC differ significantly from those previously determined indirectly, using a NADPH depletion assay, (Xue, Y., Wilson, D., Zhao, L., Liu, H.-w., Sherman, D. H. Chem. Biol. 1998, 5, 661; Lee, S. K., Park, J. W., Kim, J. W., Jung, W. S., Park, S. R., Choi, C. Y., Kim, E. S., Ahn, J. S., Sherman, D. H., Yoon, Y. J. J. Nat. Prod. 2006, 69, 847; Graziani, E. I., Cane, D. E., Betlach, M. C., Kealey, J. T., McDaniel, R. Bioorg. Med. Chem. Lett. 1998, 3117) suggesting the stoichiometric ratio between NADPH and substrate hydroxylation could not be 1:1. The presumed de-coupling between electron transfer and hydroxylation may account for this difference.

The standard reaction contains 100 nM of PikC-RhFRED (or 150 nM of wt-PikC with 3.5 μM spinach ferredoxin and 0.1 U/ml spinach ferredoxin-NADP⁺ reductase), 20˜250 μM substrate in 200 μl of desalting buffer (50 mM NaH₂PO₄, pH 7.3, 1 mM EDTA, 0.2 mM DTE, 10% glycerol). After pre-incubation at 30° C. for 5 min, the reaction was initiated by adding 1 μl of 50 mM NADPH and 50 μl aliquots were taken at 0 s, 20 s, and 40 s (or Os, 30 s, and 60 s when substrate concentration greater than 100 μM) to thoroughly mix with 2×100 μl of chloroform. The nitrogen-dried samples were redissolved in 150 μl of methanol for subsequent HPLC analysis. The HPLC conditions were: Xbridge™ C18 5 μm 250 mm reverse-phase column, 20-80% solvent B (A: deionized water+0.1% trifluoroacetic acid, B: acetonitrile+0.1% trifluoroacetic acid) at 0.8 ml/min over 20 min, UV wavelength 230 nm. The initial velocity of substrate consumption was deduced from decreased area under the curve (AUC) of specific substrate peaks. Finally, the data from duplicated experiments were fit to Michaelis-Menten equation.

### Example 6

### Liquid Chromatography Mass Spectrometry (LCMS) Analysis of in vitro Activity of Fusion Enzyme EryF-RhFRED

The EryF sample preparation was similar to that for the PikC assay (Example 5). The liquid chromatography conditions were: Xbridge™ C18 3.5 μm 150 mm reverse-phase column, 20-100% solvent B (A: deionized water+0.1% formic acid, B: acetonitrile+0.1% formic acid) at 0.2 ml/min over 20 min.

All of the above U.S. patents, U.S. patent application publications, U.S. patent applications, foreign patents, foreign patent applications and non patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

1. A chimeric protein comprising a cytochrome P450 and a heterologous reductase domain wherein the chimeric protein has self-sufficiency and increased catalytic efficiency compared to native P450 monooxygenases, said chimeric protein catalyzing hydroxylation, epoxidation, or both hydroxylation and epoxidation of a natural product substrate.

2. The chimeric protein of claim 1 wherein said substrate is a polyketide.

3. The chimeric protein if claim 1 wherein said polyketide is a macrolide.

4. The chimeric protein of claim 1, wherein the cytochrome P450 hydroxylates one or more primary, secondary or tertiary carbon atoms of said substrate.

5. The chimeric protein of claim 4, wherein the cytochrome P450 hydroxylates primary and secondary, secondary and tertiary, or primary and tertiary carbon atoms of said substrate.

6. The chimeric protein of claim 1, wherein the cytochrome P450 is naturally-occurring.

7. The chimeric protein of claim 1, wherein the cytochrome P450 is not-naturally occurring.

8. The chimeric protein of claim 1, wherein the cytochrome P450 is a bacterial biosynthetic cytochrome P450.

9. The chimeric protein of claim 1, wherein the cytochrome P450 is a mitochondrial cytochrome P450.

10. The chimeric protein of claim 1, wherein the cytochrome P450 is a eukaryotic cytochrome P450.

11. The chimeric protein of claim 1, wherein the cytochrome P450 is selected from the group consisting of PikC, EryF, MycG and TamI.

12. The chimeric protein of claim 1, wherein the heterologous reductase domain is RhFRED from Rhodococcus sp. NCIMB 9784.

13. The chimeric protein of claim 1, wherein said chimeric protein is purified.

14. A method of producing an antibiotic comprising the step of contacting a polyketide natural product substrate with the chimeric protein of claim 1 under conditions appropriate to produce an antibiotic.

15. The method of claim 14 wherein the polyketide natural product substrate is a macrolide.

16. The method of claim 14 wherein the chimeric protein is purified.
